(19) United States
(12) Patent Application Publication
(10) Pub. No.: US 2007/0258935 A1
(43) Pub. Date: Nov. 8, 2007

(54) Water dispersible films for delivery of active agents to the epidermis

(76) Inventors: Edward Enns McEntire, Kingsport, TN (US); Rebecca Reid Stockl, Kingsport, TN (US); Ramesh Chand Munjal, Kingsport, TN (US); Jessica Dee Posey-Dowty, Kingsport, TN (US); Thelma Lee Watterson, Kingsport, TN (US)

Correspondence Address: Tammye L. Taylor;Eastman Chemical Company, P.O. Box 511, Kingsport, TN, 37662-5075, US

(21) Appl. No.: 11/800,722

(22) Filed: May 7, 2007

Related U.S. Application Data
(60) Provisional application No. 60/798,574, filed on May 8, 2006.

(51) Int. Cl. A61K 8/97; A61K 8/72; A61K 36/82; A61K 36/899; A61K 36/31; A61K 36/48; A61K 36/42; A61K 36/28; A61K 36/73

(52) U.S. Cl. 424/70.11; 424/74; 424/729; 424/750; 424/757; 424/755; 424/764; 424/758; 424/765

(57) ABSTRACT

A water-dissipatable film forming formulation includes a polymer having at least one water solubilizing or dissipating moiety; an active ingredient or agent; and at least one of a plasticizer or a humectant. The present invention also includes a method for delivering an active agent to the epidermis of a subject. The method includes applying the film forming formulation to a predetermined area of skin.

### CROSS REFERENCE TO RELATED APPLICATIONS

Benefit is claimed to the earlier filed application having U.S. Ser. No. 60/798,574, filed May 8, 2006 the entire disclosure of which is incorporated herein by reference.

### FIELD OF THE INVENTION

The present invention is directed to an aqueous non-finite form composition or formulation having a cosmetic, dermatological or pharmaceutical active ingredients that when applied to the skin of a user the formulation forms a flexible, water-dispersible or water-dissipatable and/or peelable adherent film.

### BACKGROUND OF THE INVENTION

Most skin or mucosal membrane diseases or disorders, such as eczema, psoriasis, dermatitis, as well as infections from bacteria, fungal, parasitic, allergic, hormonal or other environment agents produce an inflammatory response. One important route for the administration of one or more drugs, or other active agents for treating a skin or mucosal membrane is by topical application of the active agent onto the skin. The localized treatment of body tissues, diseases and wounds requires that the particular active ingredient or agent be maintained at the treatment site for an effective period of time.

Devices for transdermal or percutaneous drug delivery are well known in the art. Such devices are typically characterized by delivering an active agent or drug to a patient's skin at a predetermined rate. Generally, such devices include a pressure sensitive adhesive containing an active component or other additive laminated onto a backing film. In some instances, the bioactive substances are mixed with and formulated into a pressure sensitive adhesive matrix which may be subsequently coated as a single pressure sensitive adhesive layer. One problem with such devices is that continuous use can lead to skin sensitization and irritation. Another problem with dermal application of active agents, and particularly with waxy film forming materials like petrolatum or petroleum jelly, is that such films are tacky and transferable when rubbed. Yet another problem with such dermal or transdermal devices is that due to the thickness and nonelasticity of the device or film, the wearer experiences discomfort during use.

Accordingly, there is still a need for a more comfortable, aesthetically pleasing, and less obtrusive topical patch or film for delivering cosmetic, dermatological, and pharmaceutical active ingredients onto the skin.

Additionally, there is a need for an active agent containing formulation that when applied to the skin, forms an adherent water-soluble, or water-dispersible, or water dissipatable, and/or peelable film on the skin, and does not dissolve due to moisture on the skin, or perspiration from the skin. Desirably, the formed active agent containing films dissolve rapidly in water, so that they may be washed off of the skin with minimum water exposure but not so delicate as to be removed by inadvertent splashing or by brief exposure to rainfall.

### BRIEF SUMMARY OF THE INVENTION

The present invention is an aqueous film forming composition of a non-finite form that forms a water-dispersible or water dissipatable, and/or peelable film on the skin for topical or transdermal application of an active agent onto or into the epidermis. The film forming composition has from about 5 to 40 weight % of a sulfonated or sulfated polymer, from about 0.001 to about 40 weight % of an active ingredient or agent, at least one of i) a plasticizer or ii) a humectant, wherein the film forming composition has a viscosity of from about 5 to about 5000 cPs, and wherein the film has a tack-free time of less than about 15 minutes.

As used herein, the term “non-finite” means that the composition is characterized by rheological properties which allow the spreading, smearing, coating, brushing, spraying or other application means when an appropriate shear force is applied to the composition. Non-limiting examples of non-finite forms include pastes, spreadable gels, lotions, emulsions, dispersions, creams, sprays, drops or ointments.

### DETAILED DESCRIPTION OF THE INVENTION

The present invention may be understood more readily by reference to the following detailed description of the invention, and to the Examples included therein.

Before the present compositions of matter and methods are disclosed and described, it is to be understood that this invention is not limited to specific methods or to particular formulations, unless otherwise indicated, and, as such, may vary from the disclosure. It is also to be understood that the terminology used is for the purpose of describing particular embodiments only, and is not intended to limit the scope of the invention.

The singular forms “a,” “an,” and “the” include plural referents, unless the context clearly dictates otherwise.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value.

Where patents or publications are referenced, the disclosures of these references in their entireties are intended to be incorporated by reference, in order to more fully describe the state of the art to which the invention pertains.

All of the substances utilized in formulating the composition of the present invention constitute known products or products which may be prepared using known techniques, some of which are commercially available. As used herein, the term “transdermal” means transdermal or percutaneous administration, i.e. application of the skin treating composition directly onto the skin to be treated. Accordingly, the terms “skin,” “derma,” “epidermis,” and the like shall also be used interchangeably unless specifically stated otherwise.

In one embodiment of the present invention, the formulation includes a film-forming material wherein the film is formed upon application of the formulation to a selected site of a user. The film is formed directly on the application site after the composition is sprayed or otherwise applied and when dry forms a film on the skin. Preferably, the dry film has a thickness of from about 0.01 mils to about 5 mils (about 0.00001 to about 0.005 of an inch). As used herein, the term “film forming” means that when the wet formulation is applied as a coating having a thickness of about 0.005 of an inch to the skin of a warm blooded mammal, it will dry to a film after two hours and will have an elongation of at least 50%.

In one embodiment of the present invention, the water-dispersible or water dissipatable film forming formulation of the present invention has from about 5 to about 40 weight % of a sulfonated or sulfated polymer; from about 0.001 to about 40 weight % of an active agent, at least one of: i) up to about 25 weight % of a plasticizer compatible with the polymer and/or ii) up to about 10 weight % of a humectant; with the remainder of the film formulation comprising water. The film forming composition has a viscosity of from about 5 to about 5000 cPs, and a tack-free time of less than about 15 minutes. The weight percentages for all the constituents are based on the total weight of the formulation.

In another embodiment the water-dispersible or water dissipatable film forming formulation of the present invention has from about 5 to about 35 weight % of a sulfonated polymer; from about 0.1 to about 30 weight % of an active agent, at least one of: i) from 0.1 to about 20 weight % of a plasticizer compatible with the polymer and/or ii) up to about 10 weight % of a humectant; with the remainder of the film formulation comprising water. The film forming composition has a viscosity of from about 5 to about 5000 cPs, and a tack-free time of less than about 15 minutes. The weight percentages for all the constituents are based on the total weight of the formulation.

In another embodiment the water-dispersible or water-dissipatable film forming formulation of the present invention has from about 10 to about 20 weight % of a polymer having at least one water-dispersible or water-dissipatable moiety; from about 1 to about 15 weight % of an active agent, at least one of: i) from about 1 to about 15 weight % of a plasticizer and/or ii) up to about 10 weight % of a humectant; with the remainder of the film formulation comprising water. The film forming composition has a viscosity of from about 5 to about 5000 cPs, and a tack-free time of less than about 15 minutes. The weight percentages for all the constituents are based on the total weight of the formulation.

In yet another embodiment of the present invention, the water-dispersible or water dissipatable film forming formulation of the present invention has from about 10 to about 20 weight % of a polymer having at least one water solubilizing or dissipating moiety; from about 1 to about 15 weight % of an active agent, at least one of: i) from about 1 to about 10 weight % of a plasticizer and/or ii) up to about 10 weight % of a humectant; with the remainder of the film formulation comprising water. The film forming composition has a viscosity of from about 5 to about 5000 cPs, and a tack-free time of less than about 15 minutes. The weight percentages for all the constituents are based on the total weight of the formulation.

The spreadable formulation of the present invention has a viscosity of from about 5 to about 5000 cPs measured at 25° C. and dries to the touch in less than about 30 minutes at 50% relative humidity. Preferably, the formulation of the present invention dries to a flexible and preferably, stretchable thin film having a thickness of less than about 5 mils (0.005 of an inch), and desirably, the film dries to the touch or is tack free in less than about 15 minutes, in another embodiment in less than about 10 minutes and in another embodiment, less than about 5 minutes. As used herein the term “dry to touch” indicates that the fluid placed on the skin no longer is wet and will no longer transfer to a dry finger that is gently pressed onto the dried film. At this time the film is also generally tack free, meaning that a clean finger when pressed on the film surface will no longer be pulled upon by the film as it is being lifted from the film surface. Tack-free time can be determined using a cotton ball test described in greater detail below. The dried film has a film thickness from about 0.2 to about 5 mils (about 5 to about 125 micrometers), and an elongation of greater than 50% as measured by ASTM method D882 for a dry film thickness from 0.6 to 0.7 mils and when evaluated following an ambient temperature cure at 50% relative humidity for 24 hours.

It is to be understood that the ranges for all constituents explicitly provided herein includes all ranges implicitly in between. For example, the range of about 5 to about 40 weight % includes about 5 to about 39 weight %, about 5 to about 38 weight %, about 5 to about 37 weight %; about to about 36 weight %, about 6 to about 40 weight %, about 6 to about 39 weight %, and so forth.

Polymers usefuil in the film forming formulation of the present invention include at least one water-dispersible or water-dissipatable moiety selected from sulfates, sulfonates, and their respective acids and salts. Many types of polymers may be used to form films containing active substances, but sulfonated or sulfated polyesters, polyacrylics, hybrid polymers having sulfonated or sulfated polyesters and mixtures thereof are particularly preferred. Both synthetic and natural polymers are suitable. These polymers should in general not be absorbable into the skin. It is desirable that the polymer have some compatibility with the active agent such that the desired amount of active agent can be incorporated into the polymer film. Suitable polymers that may be included with the sulfonated or sulfated polyester and polyacrylic include polyesters, acrylics, acrylamides, polypeptides, vinyl ether polymer and copolymers, polyalkylene glycols wherein the alkylene moiety has from 3 to 20 carbons, polyurethanes, silicones, polyalkyds, polyepoxides, polyolefins, carbohydrates, such as starches derived from different plant sources, including high amylose and high amylopectin varieties.

As used herein the term, “water dissipating moiety” means a moiety capable of dissipating a polymer in water. This is typically a polar group pendant to (attached to and protruding from) the polymer chain which allows the polymer to be dispersed or dissipated in water. The term “dissipatable polymer” means a polymer capable of being dispersed or dissipated into water because of the dissipating moiety attached to the polymer chain. When a polymer is dispersed or dissipated into water, it has a particulate nature, meaning that it has several polymer chains together in one particle. A solubilized polymer means that each polymer chain is surrounded by solvent, and polymer chains to not usually touch one another in the solution.

Other bioadhesive, water soluble polymers that may be used in conjunction with the sulfonated polyesters and polyacrylics for use in the present invention are cellulose derivatives, polysaccharide gum derivatives, polypropylene glycol, mixed polyethylene glycol-polypropylene glycol polymers—block or random copolymers, water soluble or dispersible acrylic polymers, water soluble or water dispersible polyesters, hydroxyalkyl starches, carboxymethyl starches, carboxymethyl celluloses, polyvinyl pyrrolidinone polymers and copolymers, casein, gelatin, solubilized proteins, polyacrylamide, water soluble or dispersible polyurethanes, hybrid polymers containing both acrylic and polyurethane, hybrid polymers containing both acrylic and polyester, styrene maleic ester resins, poly(olefin-maleic ester) resins and any other conventional water soluble or water dispersible polymer or a combination thereof of the above-described materials. Preferred are those polymers which are water-dispersible, rather than those truly water soluble. These polymers, when dispersed into deionized water, have a finite and measurable mean particle diameter from about 2 to about 500 nanometers or more, such as, for example, from about 5 to about 400 nanometers, or from about 10 to about 300 nanometers as measured by the Particle Size Distribution Analyzer available from Polymer Laboratories, Inc.

The polymer films may be either uncrosslinked or crosslinked. Suitable crosslinking agents that may be used are aminoplasts, di-isocyanates, polyisocyanates, polyepoxides, polyaziridines, polycarbodiimides, polyamines, polyketoesters, and the like. Polymer molecular weights (before crosslinking) that are suitable are from about 1000 to about 10,000,000 Daltons, more suitably from about 2000 to about 5,000,000. Even more suitable are polymers from about 3000 to about 1,000,000 Daltons.

Suitably, the polymer is selected from water-dispersible or water-dissipative sulfopolyesters or polyesteramides containing ether groups and sulfonate groups having a glycol residue and a dicarboxylic acid residue and at least one difunctional comonomer containing a sulfonate group attached to an aromatic nucleus and in the form of a metallic salt. Such polymers are well known to those skilled in the art and are available from Eastman Chemical Company under the trade name of Eastman AQ polyester polymers. In particular, such sulfopolyesters can be dissolved, dispersed or otherwise dissipated in aqueous dispersions, preferably at temperatures of less than about 80° C. Such polyesters are described in greater detail in U.S. Pat. No. 3,734,874 issued to Charles Kibler on May 22, 1973 the disclosure of which is incorporated herein by reference. One skilled in the art will understand that the term “residue” or “component” as used in the specification and concluding claims, refers to the moiety that is the resulting product of the chemical species in a particular reaction scheme or subsequent formulation or chemical product, regardless of whether the moiety is actually obtained from the chemical species. Thus, for example, an ethylene glycol residue in a polyester refers to one or more —OCH₂CH₂O— repeat units in the polyester, regardless of whether ethylene glycol is used to prepare the polyester. The use of the term “acid” in the above description and in the appended claims includes the various ester forming or condensable derivatives of the acid reactants such as the dimethyl esters thereof as employed in the preparations set out in these patents. Among the preferred sulfo-monomers are those wherein the sulfonate group is attached to an aromatic nucleus such as benzene, naphthalene, diphenyl, or the like, or wherein the nucleus is cycloaliphatic such as in 1,4-cyclohexanedicarboxylic acid.

Other polymers are polymers known as sulfonate stabilized water dispersible acrylic polymers are available from ALCO Chemical Company, Chattanooga, Tenn. For example, sulfonated polystyrene polymers such as VERSA-TL Sulfonated Polymers are suitable. Furthermore, partially sulfonated polystyrene polymers neutralized as alkali metal salts are suitable. These may be used alone or in conjunction with other polymers. Other such water dispersible polymers are sulfonated polystyrene polymers such as those available from National Starch under the trade name FLEXAN® II.

Other polymers suitable for use in the current invention are sulfonated or sulfated acrylic copolymers prepared from acrylamide or acrylic type monomers such as 2-acrylamido-2-methyl propanesulphonic acid (AMPS®) available from Lubrizol or sulfoethyl methacrylate (SEM) available from Polysciences, Inc. The AMPS or SEM may be polymerized with other monomers such as methyl methacrylate, butyl acrylate, styrene, and the like to form acrylic polymers. The AMPS or SEM may be present in the polymer as a salt with ammonia, an amine, or an alkali metal.

Still other suitable polymers for use in the current invention are sulfonated polymers derived from vinyl sulfonic acid and its salts, such as sodium vinyl sulfonate, available from Proviron Fine Chemicals NV of Ostend, Belgium

In accordance with the present invention, another suitable polymer is a hybrid latex of a sulfopolyester and acrylic as described in U.S. Pat. No. 6,001,922. Other examples of such sulfopolyester-acrylic hybrid polymers, wherein the acrylic monomers are polymerized in the presence of the sulfopolyester dispersion, are found in U.S. Pat. No. 4,946,932, the entire disclosures of which is incorporated herein by reference.

In regards to forming the hybrid copolymers, suitable monomers for copolymerization include but are not limited to, styrenic monomers such as styrene, alpha-methyl styrene, vinyl naphthalene, vinyl toluene, and chloromethyl styrene; ethylenically unsaturated species such as, (meth)acrylic acids and esters having carbon chain lengths of up to about 30 carbon atoms, for example, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, hexyl acrylate, hexyl methacrylate, ethylhexyl acrylate, ethylhexyl methacrylate, octyl acrylate, octyl methacrylate, fluoro or silicon containing monomers such as but not limited to octafluoropenta acrylate and trimethylsiloxyethyl acrylate, decyl acrylate, decyl methacrylate, dodecyl acrylate, dodecyl methacrylate, tridecyl acrylate, and tridecyl methacrylate, stearyl acrylate, cetyl acrylate, and the like. In addition, functional monomers such as hydroxyethyl acrylate, hydroxyethyl methacrylate, hydroxypropyl acrylate, glycidyl methacrylate, carbodiimide methacrylates such as cyclohexylcarbodiimidoethyl methacrylate; t-butylcarbodiimidoethyl methacrylate, and alkyl crotonates. Also suitable are vinyl acetate, vinyl neodecanoate, ethylene, propylene, butylene, butadiene, isoprene, di-n-butyl maleate, and di-octylmaleate; vinyl ethers such as methyl vinyl ether, butyl vinyl ether, cyclohexyl vinyl ether, sodium styrene sulfonate, sodium vinyl sulfonate, 2-acrylamido-2-methylpropane sulfonic acid or its salts, 2-sulfoethyl methacrylate or its salts; and nitrogen containing monomers including acrylonitrile, methacrylonitrile, acrylamide, methyl acrylamide, N,N-dimethyl acrylamide, methacrylamide, t-butylaminoethyl methacrylate, dimethylaminoethyl methacrylate, diethylaminoethyl methacrylate, N,N-dimethylaminopropyl methacrylamide, 2-t-butylaminoethyl methacrylate, N,N-dimethylaminoethyl acrylate, N-(methacryloyloxy-ethyl)ethylene urea and methacrlamidoethylethylene urea and mixtures thereof.

In preparing the hybrid copolymer for use in the present invention, the sulfonated or sulfated polyester or polyacrylic comprises a major component in the hybrid polymerization, and the polymerizable monomer comprises a minor component of the hybrid copolymer. In one embodiment the sulfonated or sulfated polyester or polyacrylic comprises from about 3 to about 95 weight % of the copolymer, and in another embodiment the sulfonated or sulfated polyester or polyacrylic comprises from about 5 to about 80 weight % of the copolymer, and in yet another embodiment the sulfonated or sulfated polyester or polyacrylic comprises from about 10 to about 60 weight % of the copolymer.

Plasticizers that have properties acceptable for use on the skin are useful to assist in the formation of polymer films. Plasticizers improve the adhesion of the polymer film to the skin and improve the flexibility of the polymer film. The amount of plasticizer is gauged by testing of various amounts of plasticizer in the formulation. The plasticizer preferably is at least slightly or partially soluble in water and has an affinity for the polymer in the dispersion. Plasticizers lower the Tg of the polymer as measured by differential scanning calorimetry (DSC). Thus, films containing plasticizer will have a Tg lower than the Tg of the same polymer without plasticizer. A guideline for choosing the amount of plasticizer to add is based on the polymer Tg, prior to adding any plasticizer. For example, polymers with a Tg of about 60° C., about 20 to about 25% plasticizer may be added. For polymers with a Tg of about 50° C., about 10 to about 20% plasticizer may be added. For polymers with a Tg of about 40° C., about 5 to about 15% plasticizer may be added. For polymers with a Tg of about 30° C. or less then about 0 to about 10% of a plasticizer may be added. These are only guidelines, since the exact amount of plasticizer will be dependant on the desired polymer film properties, and the exact polymer structure and the chosen plasticizer structure. Several types of plasticizers may be tested for any one polymer and the optimum plasticizer or combinations of plasticizers selected to gain the required properties for the specific application.

Plasticizers useful in this invention are generally diols, triols, polyols, alcohol ethers, alcohol esters, esters, ethers, hydroxy acids, amides, carbonates, and mixtures thereof. Suitable diols are 1,2-propylene glycol, ethylene glycol, 1,3 propylene glycol, 2-methyl-1,3-propanediol, butylene glycol, hexanediol, octanediol, and the like, containing up to 10 carbon atoms. Suitable triols are glycerin, trihydroxybutane, trihydroxyhexane, and the like. Alcohols having up to about six hydroxyl groups are suitable as plasticizers. Alcohol ethers suitable are diethylene glycol, dipropylene glycol, triethylene glycol, tetraethyene glycol, tripropylene glycol, and the like, alkoxylated alcohols such as ethoxylated alcohols, propoxylated alcohols, ethoxylated and propoxylated alcohols, where the alkoxylated alcohol is chosen from aliphatic, aromatic, alkaryl and aralkyl hydroxy functional compounds containing from 1 to 10 carbons and from one to six hydroxyl moieties. Examples of these are hydroquinone bis(hydroxyethyl ether), cyclohexanol hydroxyethyl ether, sorbitol trihydroxyethyl ether, catechol bis(hydroxyethyl ether), and mixtures thereof Suitable alcohol ester plasticizers include propylene glycol acetate, glycerin diacetate (diacetin), ethylene glycol propionate, diethyl tartrate, diethyl citrate, triethyl citrate, tributyl citrate, sorbitol tetraacetate, propylene glycol mono-octoate, and the like. Suitable esters are triacetin, acetyl triethyl citrate, acetyl tributyl citrate, dimethyl malonate, dimethyl succinate, dimethyl adipate, diethyl malonate, diethyl oxylate, ethyl benzoate, and combinations thereof.

Suitable ether plasticizers include methoxybenzene, dimethoxy benzene, diethoxy benzene, triethylene glycol dimethoxyether, and the like. Suitable hydroxy acid plasticizers include glycolic, beta-hydroxy propionic acid, lactic acid, salicylic acid, citric acid, tartaric acid, and the like. Suitable amides include alkyl formamides such as methyl formamide, dimethyl formamide, diethyl formamide, hexyl formamide, acetamide, ethyl benzamide, N,N-diethyl acetamide, N-methyl pyrrolidinone, N-ethyl beta lactam, N-methyl caprolactam, caprolactam, N,N-dimethyldecanamide and the like. Suitable carbonates include ethylene carbonate, propylene carbonate, glycerol carbonate, sorbitol bis-carbonate, and the like. Care should be taken in selecting the materials suggested above for use as plasticizers for skin contact since some may have regulatory limitations when used on the skin.

Yet another class of plasticizers may have components of one or more of the classes noted above. Non-limiting examples include polyalkylene oxides, such as polyethylene glycol, polypropylene glycol, random or block polyethylene glycol-polypropylene glycol polymers, and random or block polyethylene glycol-polybutylene glycol. Other such polymer plasticizers include polyvinyl alcohol, polyhydroxyethyl cellulose, polyhydroxypropyl cellulose, hydroxyethyl guar, polyacrylamide, polyacrylic acid, polyacrylic acid-co-maleic acid, polyacrylamide-co-acrylic acid, carboxymethyl cellulose, carboxymethyl cellulose acetate-butyrate, poly(sodium vinyl benzene sulfonate) and copolymers and combinations thereof. Amounts of these polymeric plasticizers may be from about 1 to about 10 weight percent based on the weight of the water-dispersible or water dissipatable polymer of this invention.

The humectant, if used, must be less than about 10% of the formulation. In another embodiment, the amount of humectant may range anywhere from about 0.5 to about 10% of the formulation and in another embodiment the humectant may be from about 1 and about 5% by weight of the composition. Generally, greater amounts of humectant relative to polymer may cause undesirable tackiness in the dry film, along with drying times greater than desired for skin coatings.

Humectants of the polyhydric alcohol-type can be employed as cosmetically acceptable carriers and as actives. Typical polyhydric alcohols include glycerol, diglycerol, triglycerol, polyglycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof.

A wide variety of pharmaceutical, cosmetic, skin healing, conditioning or moisturizing agents may be incorporated into the composition of the present invention as an active agent for a variety of purposes. In various embodiments, the active agent can be an anti-inflammatory, a local anesthetic, a xanthine derivative, an antihistaminic, an antifungal, an antimicrobial, an antibiotic, a cardiovascular agent, a hormone, an agent for the treatment of erectile dysfunction, a vasodilator, an analgesic, an anti-rheumatoid, an anti-itch ingredient, a chemotherapy agent, an adrenergic agonist or antagonist, an antioxidant, a moisturizing agent, an anti-hyperpigmentation agent, an anti-blotching agent, an anti-aging agent, an anti-collagenase substance, a free radical scavenger, a seboregulator, an hydrative, a keratolytic agent and an α or β-hydroxy acid, any of a variety of wound healing agents and mixtures thereof. Other active ingredients include skin lightening agents, skin nutrients, anti-sting ingredients, anti-sag ingredients, anti-cellulite ingredients, botox and alternatives, sunscreen filters, corn/wart/callus removers, absorbents, hair growth promoters, hair growth inhibitors, scalp care ingredients, sebaceous oil flow inhibitors, eye-circle care ingredients, facial mask ingredients, nail conditioning agents, skin moisturizing agents, and skin absorption promoters.

An ingredient may further have multiple functions in a formulation, for example, an emollient such as glycerol can also confer desirable physical properties to the formulation by serving also as a polymer plasticizer to provide better film formation. Another example is N,N-diethyl-m-toluamide (DEET), an insect repellant, which also serves as a plasticizer. In this latter case, the DEET residence on the skin surface is prolonged by being trapped in the dried film, and the film provides a barrier to insect bites

Optionally, the active ingredient can be formulated in an alcohol-based solvent system. In one embodiment, a lower alkyl alcohol having up to 12 carbon atoms, or up to 8 carbon atoms may be used. For example, suitable alcohols include, but are not limited to, ethanol, n-propanol, isopropanol, or an alcohol solution or suspension, such as an ethanol solution or suspension. Active ingredients such as salicylic acid, sodium bisulfite, and dl-α.-tocopherol can be prepared in the alcohol. For convenience, a formulation can be prepared using only two mixtures or solutions in which the active ingredient is dissolved in an alcohol solvent. An ingredient dissolved in an alcohol solvent may be added to the aqueous phase containing the polymer, or added to the aqueous phase containing a surfactant, whereupon the polymer may be subsequently added. The stability of the resulting formulation may be dependent on the method of preparation including the order of addition, which may be determined by those skilled in the art using routine experimentation.

In one embodiment of the present invention, the active ingredient is a topical skin-conditioning, healing or moisturizing agent. Examples of such skin-conditioning, healing or moisturizing ingredients include, but are not limited to, one or more of the following: extracts of any of aloe (for example, Aloe vera), Camellia sinensis (green tea), camomile, ginseng, grape, licorice, cucumber, corn flower, orange peel, dog rose hip, extracts from seaweed, kelp, and algae, rice bran oil, phytosterols such as dehydro-campesterol, dehydro-sitosterol, B-sitosterol, campesterol, delta-stigmasterol, brassicasterol and stigmasterol, phytosterol esters of two to thirty carbon acids, rice bran phytolipids, palm oil, squalene, coenzyme Q, erucamide, dicaprylyl carbonate, soybean or maleated soybean oil, olive oil, wheat germ oil, caffeine, carnitine, beeswax, paraffin wax, camauba wax, Shea butter, coco butter, sunflower butter, mango butter, kokaum butter, sal butter, olive butter, vegetable oil butter, glycolic acid, lactic acid, malic acid, and citric acid, salicylic acid, a polymeric hydroxylic acid, β-glucan, corticosteroids, urea, panthenol, an anthocyanidin, a phytic acid, and amino acids such as glycine, proline, lysine, leucine, alanine, arginine, and serine, avocado oil, nut and berry oils such as almond oil, walnut oil, mineral oil, petrolatum, dimethicone, dimethicone copolyol, peptides (both natural and synthetic), ubiquinone, hydroxypropyl guar, trimonium chloride, distearyl dimethyl ammonium chloride, and mixtures thereof. Moisturizing agents further include polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof. Suitable active ingredients for incorporation include extracts of natural products such as those extracted from plant or plant products by water, glycol, glycol/water blends, supercritical CO₂, glycerin, and alcohol. Desirably, the topical skin-conditioning, healing or moisturizing agent is migratory to the epidermis of the user where beneficial effects to the user of the topical skin-conditioning, healing or moisturizing agent may be derived.

Depending upon a predetermined use or benefit to be derived from the active agent, the film of the present invention, may have a fugitive active ingredient that transfers from about 1 to about 100 weight % of the active agent to the epidermis of the user. For example, in one embodiment about 10 to about 100 weight %, and in another embodiment greater than about 80 weight % of the fugitive active ingredient is transferred to the epidermis of the user.

In another embodiment of the present invention, the active ingredient may be substantially affixed within the dermatologically acceptable film upon drying so that the active ingredient is substantially non-migratory. In formulations where the active agent is non-migratory, desirably less than about 50 weight % of the active ingredient is transferred to the skin surface, for example, less than about 25 weight %, or less than about 15 weight %, or even less than about 5 weight %. An example of such an active agent is a light absorbing agent, such as an ultraviolet light absorber present in many sunscreens. These materials may be included in the polymer film by incorporating them into the polymer dispersions. These may include chemicals that absorb UVA and/or UVB radiation. These generally hydrophobic materials may be incorporated into the dispersion by a combination of heat, high shear or low shear stirring. These ingredients so incorporated may be organic or inorganic (such as titanium dioxide or zinc oxide, especially micro-fine grades with particle sizes of about 200 nanometers or less). When the polymer film dries on the skin, the UV absorbing chemicals may be held within the film and prevented from migrating into the skin, or possibly slowly released either into the skin or from the film into the environment depending on the design of the system. Suitable UV absorbers include those ingredients currently approved for use in the United States, Europe, or Japan, including cinnamates such as octyl cinnamate; benzophenones such as oxybenzone; salicylates such as 2-ethyl hexyl salicylate; benzoates such as para-aminobenzoic acid (PABA); anthranilates such as menthyl anthranilate; dibenzoyl methanes such as avobenzone; champhor derivatives such as 3-benzylidenebornan-2-one, 2-phenyl benzimidazole-5-sulfonic acid; other sunscreen chemicals such as 5-benzoyl-4-hydroxy-2-methoxy benzene sulfonic acid and 3,3′-(1,4 phenylenedimethylidene)-bis(7,7-dimethyl-2-oxo-bicyclo[2.2.1] heptane-1-methane sulfonic acid) sodium salt, mixtures of these compounds and others mentioned in Chapter 1 of “Sunscreens, Development, Evaluation and Regulatory Aspects,” edited by N. J. Lowe and N. A. Shaath, Marcel Dekker, Inc., 1990, which is incorporated herein by reference.

The formulations may further include less than about 10 weight % of a secondary beneficial ingredient such as polyvinylpyrrolidinone, silicone oils, ester emollients, cosmetically acceptable hydrocarbons, fatty acids, fatty alcohols, thickeners, humectants, preservatives, vitamins, skin lightening agents, desquamation agents, colorants, fragrances, opacifiers, abrasives and scrubbing agents. Desirably, the formulation has from about 0.001 to about 8 weight %, or from about 0.5 to about 5 weight % of the secondary beneficial ingredient.

Silicone oils may be divided into the volatile and nonvolatile variety. The term “volatile” as used herein refers to those materials which have a measurable vapor pressure at ambient temperature. Volatile silicone oils are suitably chosen from cyclic (cyclomethicone or cyclohexasiloxane) or linear polydimethylsiloxanes containing from 3 to 9, silicon atoms, such as for example from 4 to 5 silicon atoms. Nonvolatile silicone oils include polyalkyl siloxanes, polyalkylaryl siloxanes and polyether siloxane copolymers. The essentially nonvolatile polyalkyl siloxanes include, for example, polydimethyl siloxanes with viscosities of from about 5×10⁶ to 0.1 m²/sec at 25° C. Among the preferred nonvolatile emollients useful in the present compositions are the polydimethyl siloxanes having viscosities from about 1×10⁻⁵ to about 4×10⁻⁴ m²/sec at 25° C.

Ester emollients include alkenyl or alkyl esters of fatty acids having about 8 to about 30 carbon atoms, such as isoarachidyl neopentanoate, isononyl isonanonoate, oleyl myristate, oleyl stearate, isopropyl myristate, cyclohexyl 2-ethyl hexanoate, and oleyl oleate; ether-esters such as fatty acid esters of ethoxylated fatty alcohols; polyhydric alcohol esters, such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono-, di-, and tri-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters, pentaerythritol, trimethylolpropane and neopentyl glycol esters of acids having from 2 to 30 carbon atoms; esters of beeswax, spermaceti wax and tribehenin wax; sterols esters such as cholesterol fatty acid esters; sugar ester of fatty acids such as sucrose polybehenate and sucrose polycottonseedate. Also suitable emollients are fatty alcohol esters of acids such as octyl 2-ethyl hexanoate, dodecyl benzoate, hexadecyl myristate, isododecyl hydroxystearate, stearyl propionate, di-isooctyl adipate, and the like. Additional acceptable emollients are fatty ethers, fatty carbonates, and the like, where the term “fatty” indicates 8 or more adjacent or connected carbon atoms present in an alkyl chain.

Cosmetically acceptable hydrocarbons which are acceptable actives, carriers or solvents include isohexadecane, petrolatum (petroleum jelly), mineral oil, isoparaffins having from 11 to 13 carbon atoms, and polyalphaolefins, and mixtures thereof. Particularly preferred is polyvinyl pyrrolidinone having a molecular weight of from about 1000 to 100,000 Daltons. These may be used in combination with other actives, emollients, silicones, or additives.

Fatty acids having from 10 to 30 carbon atoms may also be suitable as cosmetically acceptable carriers. Illustrative of this category are pelargonic, lauric, myristic, palmitic, stearic, isostearic, hydroxystearic, oleic, linoleic, ricinoleic, arachidic, behenic and erucic acids.

Fatty alcohols having from 10 to 30 carbon atoms are another useful category of cosmetically acceptable carrier or active agent. Illustrative of this category are stearyl alcohol, lauryl alcohol, myristyl alcohol, cetyl alcohol and mixtures thereof.

Thickeners can be utilized as part of the cosmetically acceptable carrier of compositions according to the present invention. Typical thickeners include cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Also suitable are polyacrylic acids, such as those available from Noveon under the trade name Carbomer®. The class of hydrophobe modified thickeners are also suitable to modify the viscosity of the formulations. Inorganic materials may also be utilized as thickeners, particularly clays such as bentonites and hectorites, fumed silicas, and silicates such as magnesium aluminum silicate. Natural or synthetic clays may be used. Combinations of any of the above classes of thickeners may be useful.

Preservatives can desirably be incorporated into the cosmetic compositions of the invention to protect against the growth of potentially harmful microorganisms. Suitable traditional preservatives for compositions of this invention are alkyl esters of para-hydroxybenzoic acid and sorbic acid and its salts. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, and octanediol and blends thereof. Also preferred are blended products available under the trade name of Optiphen (available from International Specialty Products, Wayne, N.J. 07470). The preservatives should be selected having regard for the use of the composition and possible incompatibilities between the preservatives and other ingredients in the formulation. Preservatives are preferably employed in amounts ranging from 0.01% to about 2% by weight of the composition.

Other anti-microbial agents may also be included in the compositions of this invention. Illustrative are trichlosan, trichlocarban, Octopyrox.RTM. and zinc pyrithione. Suitably amounts may range from about 0.01 to about 5 weight %, or from about 0.1 to about 0.5 weight % of the composition.

Other active agents suitable for the compositions of the present invention include vitamins. Illustrative water-soluble vitamins are niacinamide, vitamin B₂, vitamin B₆, vitamin C, vitamin K and biotin. Among the useful water-insoluble vitamins are vitamin A (retinol), vitamin A palmitate, ascorbyl tetraisopalmitate, vitamin E (tocopherol), vitamin E acetate, retinol linoleate, vitamin C esters, tocotrienols, and DL-panthenol. Total amount of vitamins when present in compositions according to the present invention may range from about 0.001 to about 10 weight %, such as, for example, from about 0.01 to about 1 weight %, or from about 0.1 to about 0.5 weight %.

Skin lightening agents are also suitable active agents that may be included in the compositions of the invention. Illustrative substances are placental extract, lactic acid, niacinamide, arbutin, kojic acid, hydroquinone, resorcinol and derivatives including 4-substituted resorcinols and combinations thereof. Suitable amounts of these agents may range from about 0.1 to about 10%, such as, for example, from about 0.5 to about 2% by weight of the formulations.

Desquamation agents are further optional components that are suitable active agents that may be included in the compositions of the invention. Illustrative are the alpha-hydroxycarboxylic acids and beta-hydroxycarboxylic acids. Among the former are salts of glycolic acid, lactic acid, citric acid, malic acid and mixtures thereof. Salicylic acid is representative of the beta-hydroxycarboxylic acids.

Colorants, fragrances, opacifiers, chelating agents such as EDTA (ethylene diamine tetraacetic acid) and its salts and citric acid and its salts, antioxidants such as vitamin E and tert.-butyl hydroquinone, and abrasives may also be included in compositions of the present invention. Each of these substances may range from about 0.05 to about 5 weight % of the composition, such as, for example, from about 0.1 to about 3 weight %.

The film forming formulation of the present invention may further include an emulsifier. Any emulsifier suitable to obtain a stable film forming emulsion may be used. As used herein, the terms “emulsifier” and “surfactant” are considered synonymous and may be used interchangeably. The choice of appropriate emulsifier for use in this invention will depend upon the compositions of the other components selected. The selection of an appropriate emulsifier for a given system is within the purview of one skilled in the art. When present, the total concentration of the surfactant or emulsifier in the film forming compositions of the present invention may range up to about 15 weight %, such as, for example, from about 1 to about 10 weight %, or from about 1.5 to about 7 weight %, wherein the weight % is based on the total weight of the film formulation.

Suitable emulsifiers/surfactants include, but are not limited to, sodium lauryl sulfate, alcohol ethoxylate sulfate salts (an example of this class of surfactants is dodecyl alcohol 4 ethylene oxide sulfate, sodium salt), fatty alkyl alcohol ethoxylate phosphates, fatty alcohol phosphates, non-ionic alcohol ethoxylates such as hexadecanol 14 mole ethoxylate, dodecyl alcohol 20 mole ethoxylate, nonyl phenol 10 mole ethoxylate, and mixtures thereof. Both natural and synthetic surfactants may be used for stabilization of the formulation which contains the water-soluble or water-dispersible or water dissipatable polymer, plasticizer and active ingredient. Suitable natural surfactants include phosphatidylglycerol, phosphatidylcholine, such as soy lecithin, stearic acid and its salts and mixtures thereof. Suitable natural derived surfactants and emulsifiers include cholesterol ethoxylates, glyceryl stearate, vitamin E ethoxylate, glyceryl, diglyceryl, polyglyceryl monoesters of fatty acids, ester ethoxylates such as d-alpha-tocopheryl Polyethylene Glycol-100 Succinate (also known as Vitamin E-TPGS (available from Eastman Chemical Company)), and the like. In the above paragraph, fatty refers to a saturated aliphatic hydrocarbon group of about ten or more joined carbon atoms, or an aryl or alkaryl hydrocarbon group of about 10 or more joined carbons.

Other surfactants or emulsifiers may be selected from the group consisting of anionic, nonionic, cationic and amphoteric materials. In one embodiment, suitable nonionic surfactants are those having a C₁₀ to C₃₀ fatty alcohol or fatty acid hydrophobe condensed with from 2 to 100 moles of ethylene oxide per mole of hydrophobe; C₂ to C₁₂ alkyl phenols condensed with from 2 to 50 moles of alkylene oxide; fatty acid monoglyceride; sorbitan, mono- and di- C₈ to C₂₀ fatty acid sorbitan esters; and polyoxyethylene sorbitan esters as well as combinations thereof.

Suitable anionic surfactants include, but are not limited to, alkyl ether sulfates and sulfonates, alkyl sulfates and sulfonates, alkylbenzene sulfonates, alkyl and dialkyl sulfosuccinates, C₈ to C₂₀ acyl isethionate, C₈ to C₂₀ alkyl ether phosphates, C₈ to C₂₀ sarcosinates and combinations thereof.

Suitable natural surfactants include, but are not limited to, lecithin and lecithin derivatives (available from ADM Company, Decatur, Ill. 62526 as Standard Lecithins, Thermolec® Lecithins and Ultralec® Deoiled Lecithins). Other suitable natural surfactants are monoglycerides derived from soy, palm, and vegetable sources, (available from ADM under the trade name of Panalite®). Other suitable phospholipids include synthetic phosphate esters such as dicetyl phosphate and cetyl phosphate, ceteth-10 phosphate and blends, under the trade name of Crodafos®, such as Crodafos-CES, -CS20 Acid, -CP50, and -HCE, (available from Croda, Inc, Edison, N.J. 08837-3907). Alkyl polyglycosides and saccharide fatty amides (e.g. methyl gluconamides) are also suitable nonionic surfactants.

In another embodiment of the present invention, a method for delivering an active agent to the epidermis or skin is provided. The method includes the steps of preparing the formulation of the present invention having the desired active ingredient, and applying the formulation to a predetermined area of skin. Advantageously, by incorporating a desired concentration of active agent in the formulation, it is possible to administer varying amounts of the active agent to the subject, (person, mammal or reptile) by changing the size of the contact area.

Compositions of the present invention may be in any non-finite form such as a lotion, cream, roll-on formulations, sticks, mousses, aerosol and non-aerosol sprays. Accordingly, the formulation can be delivered for use in a variety of devices, such as a rollette applicator, ajar having an apical manual pump, sprayed using an atomizer, or spread directly from a tube or bottle. A “rollette” applicator is a ball-tipped container such as is commonly used for application of deodorant. A “jar having an apical manual pump” includes a container capable of using compressed air produced by manual depression and release of a movable piston, which imparts to the compressed air a volume of the composition described herein for delivery to the skin of the subject. A “tube” is a compressible delivery container having a cap or cover, such as the ones typically used for the delivery of topically acting active agents in the form of, for example, creams, ointments, gels and pastes.

Desirably, the effective glass transition temperature, Tg, of the dried polymer film formed from the formulation should be from about −40 to about +40° C., such as, for example, from about −30 to about +30° C., or even from about −25 to about +25° C. By “effective glass transition temperature” we mean the polymer phase transition from the glassy phase to the rubbery phase as measured by differential scanning calorimetery, after the addition of active, plasticizer, tackifier, surfactant, solvent, or other ingredients to the formula. The Tg is the mid-point of a temperature range in which the polymer gradually becomes more liquid (less viscous) and changes from being solid and glassy to elastomeric and rubbery. Additives such as active ingredients, plasticizers, tackifiers, surfactants, solvents, and the like often have the effect of reducing the effective Tg of the film forming polymer.

The term “dried film or dried polymer film”, means when most of the solvent (water and/or other solvent) has evaporated as defined by the film drying time of 2 hours at 32° C. for a wet film thickness of 5 mils of applied formula as applied to a non-porous smooth substrate such as glass or metal under the drying conditions of 50% ambient humidity with no forced air currents during the drying process. At this 2 hour drying time, the film has gained most of its ultimate film properties. The time at which the drying film has achieved its ultimate properties, as defined for proposes of this invention, is the drying time of 24 hours at 50% relative humidity and at 22 to 25° C.

In preparing the formulation of the present invention, an aqueous first mixture comprising the water-dissipatable polymer, at least a portion of the water and a plasticizer, if used, is prepared. A second mixture is then prepared consisting of an oily phase wherein the solid, oily or waxy ingredients are combined and liquefied. Heat may be added if needed to liquefy the solid, oily or waxy ingredients. Desirably, the solid or waxy ingredients are first melted or softened or dissolved in a suitable oil or solvent prior to adding to the first mixture. The combined mixture, or third mixture, is then homogenized using a high speed shear until the second mixture has cooled

In another embodiment, the formulation of the present invention can be prepared by making a first mixture comprising the softened solid or waxy ingredients, an emulsifier and optionally a surfactant. The aqueous phase comprising the water-dissipatable polymer dispersed in water is then added, while mixing, to the first mixture to make a second mixture. Homogenization may be used as above.

In yet another embodiment, all the ingredients are blended together. Water and/or other liquids are then added to the blend and to make a first mixture. The mixture is then stirred and, if necessary, heated and/or homogenized to form a uniform second mixture.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### Standard Conditions for Measuring Drying and Film Forming Ability of a Formulation.

Since determining formulation and film properties is problematic when a film resides on skin, a suitable substitute method was devised and is presented below. These tests will allow laboratory measurement of properties in a reproducible fashion.

Viscosity of the polymeric composition is determined using an AR 2000 parallel plate rheometer available from TA instruments, New Castle, Del., and measuring at a shear rate of 100/sec.

A film was applied at the uniform thickness using a 5 mil gap (125 micrometers) applicator, 8-Path Wet Film Applicator available from the Paul N. Gardner Company, Inc., Pompano Beach, Fla. applied a 2 inch wide film to a flat metal substrate at the controlled substrate temperature of 32° C. and the ambient air temperature was controlled at 22-25° C. in a location having present no forced air currents that contact the drying film. The relative humidity was controlled at 45-55%. The wet film thickness measured immediately following the drawdown was typically from 3 to 3.5 mil. The term “wet film thickness” means that thickness of the freshly applied wet film before drying as measured by any of the wet film thickness gauges typically used in the coatings industry, such as the Nordson-Gardco wet film thickness gauge available from the Paul N. Gardner Company, Inc., Pompano Beach, Fla.

To control the substrate surface temperature a Rhopoint Minimum Film Forming Temperature Bar (available from the Paul N. Gardner Company, Inc., Pompano Beach, Fla.) was used. The location of the portion of the metal bed which was at 32° C. was marked with a solvent soluble marker. A wet film was applied with a two inch wide applicator across the bed so that the 32° Centigrade portion of the bed was completely covered by the drawdown and the location mark bisects lengthwise the drawdown drying film. The lid of the instrument was left open during the test.

The “tack-free time” is measured during drying of the film. It is defined as the time starting from the film formation (drawdown) to when, at 32° C. substrate temperature, the film no longer acquires fibers from a cotton ball (having a weight of 0.6 to 0.8 grams) being slowly rolled across the width of the drying film. This time corresponds approximately to the time at which the film, when pushed gently with a clean finger, no longer pulls on the finger as the finger was withdrawn. In accordance with the present invention, the film will have a tack free time of 15 minutes or less, and in a preferred embodiment of less than about 5 minutes.

The “dry-to-touch” time is the time from when the drawdown until when the film is gently probed with a clean finger and the finger has no material transferred from the drying film.

Elongation: The elongation was measured on a free film (not in contact with a substrate or surface) according to the procedure of ASTM Method D882. The free film was prepared at a dry film thickness of 0.6 to 0.7 mil (0.0006 to 0.0007 inches thick) by making a drawdown with a suitable applicator on a release substrate. The release substrate may be any of a variety of substrates, so long as it is flat and non-porous, and have a low surface energy so that the film, when dry, may be readily separated from the substrate. Suitable substrates used include poly(tetrafluoroethylene), siliconized polyester film, siliconized paper, and wax paper. Drying conditions for this test were 22-25° C. air and substrate temperature for 24 hours. Afterwards the dried film was removed from the substrate and an elongation measured. In accordance with the present invention, the film must have an elongation of at least 50% and preferably is greater than 200%, when the sample was pulled at a rate of 10 inches per minute.

### COMPARATIVE EXAMPLE 1

A film forming gel was prepared as stipulated in Example 1 of U.S. Pat. No. 4,950,475 by placing about 40 weight % propylene glycol and 30 weight % water in a suitable container and heating the mixture to 80° C. The heated aqueous phase produced was transferred to a blender and 30 weight % Eastman AQ 55S polymer (available from Eastman Chemical Company) was gradually added with the blender providing high shear mixing to dissolve the solids. When all the solids were added, high shear mixing was continued until a clear, straw colored thick gel was produced. The gel was cooled to room temperature.

The drying time of the gel was evaluated by placing 0.11 grams of the gel over a 4 cm² area of the volar forearm of a test subject. The gel took 52 minutes to dry to a point that the gel was not wet to the touch and over 180 minutes for the gel to be tack free.

### EXAMPLE 2

Preparation of Sulfopolyester A

A round bottom flask equipped with ground-glass head, an agitator shaft, nitrogen inlet and a side arm was charged with 82 mole percent isophthalic acid, 18 mole percent dimethyl-5-sodiosulfoisophthalate (SIP), 54 mole percent diethylene glycol (DEG), and 46 mole percent 1,4-cyclohexanedimethanol (CHDM), based on 100 mole percent dicarboxylic acid and 100 mole percent diol. A catalyst was added and the flask was immersed in a Belmont bath at 200° C. for one hour under a nitrogen sweep. The temperature of the bath was increased to 230° C. for one hour. After one hour the temperature of the bath was increased to 280° C. and the flask was heated for 45 minutes longer under a reduced pressure of 0.5 to 0.1 mm of Hg. The flask was allowed to cool to room temperature. The copolyester was removed from the flask and ground to less than 3 mm granules. Sulfopolyester A had a Tg of 53° C. (as determined by differential scanning calorimetery) and an Inherent Viscosity (I.V.) of 0.33 dl/g was measured at 23° C. using 0.50 grams of polymer per 100 ml of a solvent consisting of 60% by weight phenol and 40% by weight tetrachloroethane.

A dispersion of the Sulfopolyester A polymer granules was prepared by heating to 80° C. 136 grams of deionized water in a 500 milliliter beaker. Then 64 grams of the polymer granules were added with stirring, and the stirring continued for 30 minutes. The weight of the water that evaporated on heating was replaced as the formula cooled, giving a nearly clear polymer dispersion.

### EXAMPLE 3

### Preparation of Sulfopolyester B

A round bottom flask equipped with ground-glass head, an agitator shaft, nitrogen inlet and a side arm was charged with 78.0 mole percent isophthalic acid, 22.0 mole percent dimethyl-5-sodiosulfoisophthalate (SIP), 77.0 mole percent diethylene glycol (DEG), and 23.0 mole percent 1,4-cyclohexanedimethanol, based on 100 mole percent dicarboxylic acid and 100 mole percent diol. A catalyst was added and the flask was immersed in a Belmont bath at 200° C. for one hour under a nitrogen sweep. The temperature of the bath was increased to 230° C. for one hour. After one hour the temperature of the bath was increased to 280° C. and the flask was heated for 45 minutes longer under reduced pressure of 0.5 to 0.1 mm of Hg. The flask was allowed to cool to room temperature and the copolyester was removed from the flask. The sulfopolyester was ground to less than 3 mm granules. Sulfopolyester B has a Tg of 47° C. and an I.V. of 0.33 dl/g using 0.50 grams of polymer per 100 ml of a solvent consisting of 60% by weight phenol and 40% by weight tetrachloroethane.

A dispersion of Sulfopolyester B polymer granules was prepared by heating to 80° C. 136 grams of deionized water in a 500 milliliter beaker. Then 64 grams of the polymer granules were added with stirring, and the stirring continued for 30 minutes. The weight of the water that evaporated on heating was replaced as the formula cooled, giving a nearly clear polymer dispersion.

### EXAMPLE 4

### Preparation of Sulfopolyester C.

Following the procedure of Example 2 above Sulfopolyester C was prepared with the following exceptions: 11 mole percent dimethyl-5-sodiosulfoisophthalate and 89 mole percent isophthalic acid, and 21.5 mole percent 1,4-cyclohexanedimethanol and 78.5 mole percent diethylene glycol, based on 100 mole percent dicarboxylic acid and 100 mole percent diol. The resultant Sulfopolyester C has a Tg of 35° C. and an I.V. of 0.32 dl/g using 0.50 grams of polymer per 100 ml of a solvent consisting of 60% by weight phenol and 40% by weight tetrachloroethane.

A dispersion of the Sulfopolyester C polymer granules was prepared by heating to 80° C., 136 grams of deionized water in a 500 milliliter beaker. Then 64 grams of the polymer granules were added with stirring, and the stirring continued for 30 minutes. The weight of the water that evaporated on heating was replaced as the formula cooled, giving a slightly turbid polymer dispersion.

### EXAMPLE 5

### Preparation of Sulfopolyester D

A 1000 mL round bottom flask equipped with a ground-glass head, agitator shaft, nitrogen inlet, and a sidearm was charged with 184.0 grams (0.92 moles) of dimethyl cyclohexanedicarboxylate, 23.7 grams (0.08 mole) dimethyl-5-sodiosulfoisophthalate, 95.4 grams (0.90 mole) diethylene glycol, 43.2 grams (0.30 mole) 1,4-cyclohexane dimethanol, 6.70 grams (0.05 mole) trimethylol propane, and 1.17 mL of a 1.46% (w/v) solution of titanium isopropoxide in n-butanol. The flask was purged with nitrogen and immersed in a Belmont metal bath at 200° C. for 90 minutes and 220° C. for an additional 90 minutes under a slow nitrogen sweep with sufficient agitation. After elevating the temperature to 280° C., a vacuum of less than or equal to 0.5 mm of Hg was installed for 10 minutes to perform the polycondensation. The vacuum was then displaced with a nitrogen atmosphere and the polymer was allowed to cool after removing the flask from the metal bath. Sulfopolyester D had an inherent viscosity of 0.210 dL/g as determined according to ASTM D3835-79 and a glass transition temperature of −4° C. obtained using thermal analysis by DSC. The polymer was clear and nearly colorless

A dispersion of the Sulfopolyester D polymer in water was prepared by heating to 80° C., 160 grams of deionized water in a 500 milliliter beaker. Then 40 grams of the polymer was added with stirring, and the stirring continued for 30 minutes. The weight of the water that evaporated on heating was replaced as the formula cooled, giving a slightly turbid polymer dispersion

### EXAMPLE 6

In accordance with the present invention, a formulation was prepared by combining in a 1 ounce wide-mouth jar the following constituents: (a) 20.44 g of the dispersion from Example 2; (b) 1.2 g triacetin (available from Eastman Chemical Company); (c) 1.2 g DG Petroleum Jelly (available from Dolgen Corp., Inc., 100 Mission Ridge, Boodlettsville, Tenn. 37072); and (d) 0.47 g Clearate Lecithin emulsifier (available from W.A. Cleary Corp., 1049 Route 27, P.O. Box 10, Somerset, N.J. 08875-0100). The bottle was placed in a water-bath at 80° C. for 1 hour. The bottle was removed, and was shaken rapidly on a Brinkman Vibratory Mill until it was cool. The emulsion was creamy and did not separate upon standing.

The formulation formed a film in less than 5 minutes when an amount was brushed to the back of a test subject's hand and allowed to dry. The film was not greasy to the touch, and was not tacky to the touch after the 5 minutes drying time. After 2 hours, the film was removed from the test subject's hand by washing with water. The skin beneath the spot where the film had resided felt smooth to the touch.

The formulation was drawn down on a release film (Polyester Liner L-25X available from Sil-Tech, 222 Mound Avenue, Miamisburg, Ohio 45342) using a 4 mil (0.004 of an inch) gap film applicator which deposited an approximately 2 mil (0.002 inch) thick wet film. The film was allowed to dry at ambient temperature overnight. The dry film, before elongation, had a thickness of 0.66 mil (0.00066 of an inch). The dry coating had an elongation of greater than 600% as measured by ASTM Method D882, demonstrating the high flexibility of the film.

### EXAMPLE 7

A formulation was prepared by combining under high shear conditions, 28.1 weight % of the polymer from Example 2 dispersed in 59.6 weight % water, 5.15 weight % triacetin, 5.15 weight % petrolatum and 2 weight % lecithin. When all the solids were added, high shear mixing was continued until a stable dispersion was produced.

The drying time of the liquid was evaluated by spreading 0.12 grams of the liquid formulation over a 4 cm² area of the volar forearm of a test subject. The formulation took 9 minutes to dry tack free, even with the film having a thickness of approximately 4.8 mils (0.12 millimeters).

### EXAMPLE 8

A test sample of 0.0036 grams of petrolatum was spread over a 4 cm² area of the volar forearm of a test subject. The petrolatum did not dry or become tack free within one hour.

### EXAMPLE 9

In accordance with the present invention, a formulation was prepared by combining in a 1 ounce wide-mouth jar the following constituents: (a) 20.44 g of the dispersion from Example 2; (b) 1.2 g glycolic acid; (c) 1.2 g DG petroleum jelly; and (d) 0.47 g Clearate Lecithin emulsifier. The bottle was placed in a water-bath at 80° C. for 1 hour. The bottle was removed, and was shaken rapidly on a Brinkman Vibratory Mill until it was cool. The emulsion was thick and did not separate upon standing.

The emulsion formed a film in less than 5 minutes when it was applied by the hand to the skin. The film was not greasy or tacky to the touch. After 2 hours, the film was removed from the hand by washing with water. The skin beneath the film felt smooth to the touch.

### EXAMPLE 10

In accordance with the present invention, a formulation was prepared by combining in a 1 ounce wide-mouth jar the following constituents: (a) 17.79 g of the dispersion from Example 2; (b) 1.04 g NutriLayer® phytolipids (a rice bran product available form Eastman Chemical Company); and (c) 1.04 g coconut oil fatty acid (COFA). The bottle was placed in a water-bath at 80° C. for 4 hours. The bottle was removed, and shaken rapidly on a Brinkman Vibratory Mill until it was cool. The emulsion was thin and stable to separation. To this first mixture was added the following: (d) 0.74 g glycerin and (e) 0.37 g glycolic acid. The bottle was placed in a water-bath at 80° C. for 1 hour. The bottle was removed, and was shaken rapidly on a Brinkman Vibratory Mill until it was cool. The emulsion was creamy and did not separate upon standing.

The mixture formed a dry film in less than 3 minutes when it was applied to the hand with a small brush. The film was flexible, not greasy nor tacky to the touch. After 2 hours, the film was removed from the hand by washing with water. The skin beneath the film felt smooth to the touch.

### EXAMPLE 11

In accordance with the present invention, a formulation was prepared by combining in a 1 ounce wide-mouth jar the following constituents: (a) 9.38 grams of the dispersion from Example 3; (b) 9.38 grams of the dispersion from Example 4; (c) 0.75 grams triacetin; (d) 0.75 grams of an emulsifier (Crodafos CES (Croda, Inc.)); (e) 0.75 grams of Thermolec 57 lecithin (available from ADM Company); (f) 2.7 grams of petroleum jelly (Vaseline®, available from Unilever); and (g) 6.3 grams of water. The bottle was placed in a water-bath at 85° C. for 1 hour. The bottle was removed, and was shaken rapidly on a Brinkman Vibratory Mill until it was cool. The emulsion was thick and stable to separation.

### EXAMPLES 12 AND 13

Following the procedure used in EXAMPLE 11, blends of the materials specified and in their respective amounts shown in Table 1 below were prepared by shaking as in Example 11 after heating to 90° C. for about one hour. Films were prepared by ambient temperature curing a 5 mil wet drawdown overnight.

TABLE 1

| | | Polymer | NutriLayer | | |
|---|---|---|---|---|---|
| Ex. | Polymer | Dispersion | Phytolipid | Water | |
| no. | Name | wt (g) | wt (g) | wt (g) | Results |
| 12 | PolyAMPS | 23.63 | 1.56 | 0 | Dispersion of |
| | EXP-4888^{a}, | | | | limited stability |
| | 33% | | | | gave a clear, |
| | | | | | brittle film |
| 13 | Versa-TL 501^{b}, | 20.13 | 1.48 | 0 | Translucent |
| | 25% and | 6.84 | | | dispersion gave |
| | Foral AX- | | | | a translucent, |
| | E Dispersion, | | | | non-tacky |
| | 35%^{c} | | | | flexible film |

^{a}A sodium salt dispersion of an acrylic polymer available from ALCO Chemical Company, Chatanooga, TN 37406.
^{b}A 25% solution of a sulfonated polystyrene, sodium salt, dispersion available from ALCO Chemical Company, Chatanooga, TN 37406.
^{c}Foral AX-E is a fully hydrogenated rosin available from Eastman Chemical Company.

### EXAMPLE 14

In accordance with the present invention, a formulation that provides a film on the skin which contains an ultraviolet light absorber was prepared by combining the following constituents: (a) 47.7 grams of the dispersion of Example 2; and (b) 2.61 g of triethyl citrate. These were blended together to produce 50.3 g of a polymer-plasticizer blend. To this was added (c) 4.08 grams of ethylhexyl methoxycinnamate (Uninul® MC-80, available from BASF, Inc). After additional blending and heating to 60° C. for one hour, the blend was then cooled. A viscous, slightly opaque dispersion was produced.

When the dispersion was applied to the volar forearm of a test subject at the rate of 0.01 grams per square centimeter using a small brush, and the liquid dried rapidly (in less than five minutes) to a film. The dry film remained on the skin for 3 hours, and was then removed, collected completely and analyzed by liquid chromatography for ethylhexyl methoxycinnamate. The film retained most (69-78% in two experiments) of the ultraviolet light absorbing chemical.

Conversely, when the active, ethylhexyl methoxycinnamate, was applied to the skin at nearly twice the dosage to that above, the entire amount of active ingredient was absorbed into the skin within one hour. This demonstrates that much less of an ultraviolet light absorbing chemical is absorbed into the skin when the chemical is contained in the dry film, even though the formulation is applied as a liquid.

### EXAMPLE 15

A film forming formulation was prepared by adding 1.0 gram salicylic acid to 50 g of a dispersion prepared according to that of Example 2. The formulation was blended by rolling the bottle containing the components on a bottle roller. The viscosity of this formulation increased significantly from the viscosity of the original dispersion before the acid addition. The increase of viscosity indicates that the salicylic acid was plasticizing the polymer in that it was absorbing into the particle. A clear even film resulted from a drawdown on glass with a 3 mil gap draw bar.

### EXAMPLE 16

A film forming formulation was prepared by adding 1.0 gram of dipropylene glycol dibenzoate, (Benzoflex 9-88, available from Velsicol Chemical Corporation, Rosemont, Ill. 60018-3713) to 50 grams of a dispersion prepared according to that of Example 2. The material was blended by rolling the bottle containing the components on a bottle roller. The viscosity of this formulation increased significantly from the viscosity of the original dispersion before the addition of the Benzoflex 9-88. A sample of the blend was drawn down with a 3 mil gap draw bar on a glass surface. A clear dry film resulted, but the film was still brittle when removed from the glass by scraping with a sharp blade. This indicates that this concentration of plasticizer was too low because the film was brittle.

When 8 grams of Benzoflex 9-88 was added to 50 g of a dispersion prepared according to that of Example 2, the resulting film was flexible but cloudy. This indicates that this concentration of plasticizer was too high because phase separation was evident in the drawdown.

### EXAMPLE 17

The dispersion of Example 2 was drawn down using the 3-mil gap applicator. The resulting dry film had a thickness of approximately 1.2 mils (about 30 micrometers), was clear, but brittle in that it turned to a powder when scraped from the glass substrate with a sharp blade. This example illustrates the need to add a plasticizer to films having a Tg of 53° C.

### EXAMPLE 18

A formulation was prepared having: (a) 335.24 grams of a dispersion prepared according to that of Example 2; (b) 20.74 grams of triacetin; and (c) 10.56 grams of Thermolec 57 lecithin, (available from ADM Company). The material was blended together using high shear mixing until homogeneous. The blend was heated to about 55° C. from the mixing with the Ultra-Turrax T50 disperser. Then (d) 26.91 grams of petrolatum and (e) 4.17 grams of a 2% solution of EDTA disodium salt, were added. The blend was heated without stirring to 90° C. for 1.25 hours. While cooling, the blend was subjected to the Ultra-Turrax high shear mixing again for 5-10 minutes. The blend was then stirred with low shear mixing until cool.

### EXAMPLE 19

A formulation was prepared by blending: a) 335.24 grams of a 32% aqueous dispersion of the polymer of Example 2; b) 20.74 grams triacetin; c) 10.56 grams lecithin; d) 26.91 grams petrolatum; and e) 4.17 grams of a 2% aqueous solution of EDTA.2Na.H₂O. When all the solids were added, high shear mixing was continued until a stable dispersion was produced. To 75.25 grams of this blend was added 4.01 grams of ACEMATT® OK412 precipitated silica, (available from Degussa) which was stirred in by hand using a wooden tongue depressor. When the viscous blend was applied to the skin on the wrist or knuckles, the dried film was not visually apparent. The system appeared to fill wrinkles so that their appearance was diminished. It was also observed that resistance of the dry film to crack on the skin was improved relative to an identical film without the silica. A drawdown using a bar with a 3 mil gap of the same mixture on an aluminum Q-panel gave a 60° gloss measurement of 10.2, versus the gloss of the control film of 62.6, indicating that a substantial reduction in film gloss has occurred by adding the silica.

### EXAMPLE 20

Fifty (50) grams of a polymer dispersion prepared according to Example 2 was combined with 4 grams glycolic acid and 1 gram of Thermolec 57 lecithin. The dispersion was drawn down on a flat glass substrate with a 5 mil gap applicator, and the resulting film was evaluated after drying at room temperature overnight. The film was clear but contained cloudy spots, yet was very flexible and strong. The elongation was estimated by stretching the film to at least 100% elongation.

### EXAMPLES 21-23

The following ingredients were placed in separate one ounce wide-mouth jars:

| Example 21 | Example 22 | Example 23 |
|---|---|---|
| 25.0 g Dispersion of Ex. 2 | 25.11 g | 25.06 g |
| | Dispersion of Ex. 3 | Dispersion of Ex. 4 |
| 0.8 g Glycerin | 0.8 g | 0.8 g |
| | Glycerin | Glycerin |
| 0.4 Glycolic Acid | 0.4 | 0.4 |
| | Glycolic Acid | Glycolic Acid |

Each Example was placed in a water-bath at 85° C. for 1 hour. The bottles were removed, and were shaken rapidly on a Brinkman Vibratory Mill until they were cool. These Examples were drawndown on glass using a 5 mil gap applicator. The resulting air-dried films were very clear and flexible with good elongation and recovery.

### EXAMPLE 24

The following ingredients were placed in a one ounce wide-mouth jar: (a) 11.8 g sulfopolyester dispersion of Example 3; (b) 0.38 g glycerin; (c) 0.19 g glycolic acid; and (d) 2.0 g NutriLayer Phytolipid (Eastman Chemical Company). The bottle was placed in a water-bath at 85° C. for 1 hour. The bottle was removed, and was shaken rapidly on a Brinkman Vibratory Mill until it was cool. The emulsion was thin and stable to separation.

The mixture formed a dry film in less than 2 minutes when it was applied by a fine brush to the hand. The dry film was flexible, not greasy, and not tacky to the touch. The film was removed by washing with water. The skin beneath the film was smooth. Translucent films made by applying this emulsion to glass using a 5 mil gap applicator and allowing to air dry, were tough with good elongation and recovery.

### EXAMPLE 25

The following ingredients were placed in a one ounce wide-mouth jar: (a) 17.81 grams of Sulfopolyester Dispersion of Example 2; (b) 1.04 grams of NutriLayer Phytolipid (Eastman Chemical Company); (c) 1.04 g of COFA; (d) 0.74 g of glycerin; and (e) 0.37 g of glycolic acid. The bottle was placed in a water-bath at 85° C. for 1 hour. The bottle was removed, and shaken rapidly on a Brinkman Vibratory Mill until it was cool. The emulsion was creamy and stable to separation.

The mixture formed a dry film in less than 2 minutes when it was applied by a fine brush to the hand. The dry film was flexible, not greasy or tacky to the touch. The dry film was removed by washing with water. The skin beneath the film was smooth. Translucent films made by applying this emulsion to glass using a 5 mil gap applicator and allowing to air dry, were tough with good elongation and recovery.

### EXAMPLE 26

A polyester-acrylic hybrid which is 2:1 weight ratio of sulfopolyester A:(80/20 methyl methacrylate/butyl methacrylate copolymer, polymerized in the presence of Sulfopolyester A was prepared as described in Example 1 of U.S. Pat. No. 4,946,932, with the following exceptions: Sulfopolyester A is substituted for the sulfopolyester in Example 1 of U.S. Pat. No. 4,946,932; the monomer mixture above was substituted for the monomer mixture described in Example 1 in U.S. Pat. No. 4,946,932; and the ratio of Sulfopolyester A and the acrylic monomers were adjusted to proportions to achieve a 2:1 mass ratio of sulfopolyester to acrylic polymer.

The following ingredients were placed in a one ounce wide-mouth jar: (a) 15.6 g of the polyester-acrylic hybrid having 40% solids; (b) 1.56 g of petrolatum; (c) 1.04 g of Thermalec 57 lecithin (Archer Daniels Midland Company); and (d) 0.94 g of triacetin (Eastman Chemical Company). The bottle was placed in a water-bath at 85° C. for 1 hour. The bottle was removed and shaken rapidly on a Brinkman Vibratory Mill until it was cool. The emulsion was thin and stable to separation. Films made by applying this emulsion to glass using a 5 mil gap applicator and allowing to air dry, were waxy and brittle.

### EXAMPLE 27

A water-dispersible hybrid miniemulsion containing 0.6 part of AQ48 polyester (Eastman Chemical Co) and 1 part of monomer mix (Tg of −5° C.) and 20 weight % Petrolatum was prepared as follows:

Water (535 grams ) and AQ 48 (230 grams) polyester pellets were added to a 2000 mL resin reactor equipped with a condenser, nitrogen purge, and a subsurface feed tube. A nitrogen purge was initiated and the contents were heated to 80° C.

A monomers premix was prepared having:

1. 345.0 grams of 2-ethylhexyl acrylate/methacrylic acid in a 65/35 weight ratio,

2. 300 grams of water, and

3. 15.5 grams of a surfactant blend having Aerosol OT-NV (available from Cytec Industries) and/or Hitenol BC1025 (available from DKS) in ratio of 1.1:0.4.

Petrolatum (69 grams), purchased as Petroleum Jelly, was slowly added to a monomers premix and stirred for 3 hours to obtain a milky looking dispersion. The dispersion was sheared using an IKA (Model SD-45) rotor/stator homogenizer by pumping the dispersion through a flow cell operating at maximum rpm to form a miniemulsion.

A reaction initiator feed was prepared having 90.0 g of water, 1 g of ammonium persulfate, and 1 g of ammonium carbonate.

Ammonium persulfate (0.65 g) was mixed in 12 g of water and charged to the reactor mixture. After 1 minute, the miniemulsion was fed to the reactor over a period of 180 minutes. Concurrently with the miniemulsion feed, the initiator feed was also fed to the reactor but over a time period of 195 minutes.

After the feeds ended, the reactor was held at 80° C. for 60 minutes, then cooled to 50° C. A reductant solution consisting of 10 g water, 1.0 g isoascorbic acid, and 1.2 g of 0.5% iron sulfate heptahydrate, and 0.34 g of 28 weight % ammonium hydroxide was added to the reactor.

A solution of 25.0 g water and 1.2 g 70 weight % t-butyl hydroperoxide was then fed to the reactor over a period of 48 minutes. The reaction product was then cooled to room temperature. The resulting latex was filtered through a 100 mesh wire screen and filterable solids (scrap) was determined as less than 0.1 weight %, based on the total batch weight. Mean particle size of the finished latex was 262 nm. The droplet and latex particle sizes were measured using Microtrac UPA Particle Size Analyzer laser light-scattering device (180° backscattering). To determine particle size, the sample was diluted in water at a ratio of approximately 1:50 v/v. The resulting miniemulsion latex was drawn down to form a film on glass, then heated for 5 minutes at 80° C. to drive off the water. The film was found to be readily removable from the glass surface with mild rubbing with water.

### EXAMPLE 28

This example illustrates a composition wherein a secondary beneficial additive is included which limits cracking of the film when applied to the skin.

Aqueous Phase: The following ingredients were sequentially added, with mild stirring, to a beaker while heating to 75° C.: 1) 71.4 g of a 32% solids dispersion of Eastman AQ55 polyester; 2) 11.3 g deionized water; 3) 1.83 g glycerin; 4) 0.57 g of polyvinylpyrrolidinone polymer (Mw 90,000 Daltons); 4) 0.05% disodium EDTA dehydrate; and 5) 0.76 grams cetyl phosphate, potassium salt (Amphisol K, available from DSM);

Organic Phase: In a separate beaker were added the organic phase ingredients, also with stirring and heating to 80 to 90° C.: a) 5.00 g Petrolatum; b) 2.29 grams triethyl citrate; c)1.52 g cetearyl alcohol (Lanette O available from Cognis); and d) 1.00 g Glycerol monostearate (Cutina GMS V, available from Cognis).

The organic phase was then poured into the aqueous phase with stirring. Then 4.00 g of Silica MSS-500/3H4 (available from Kobo Products, Inc., Plainfield, N.J., USA) was added with stirring. The entire mixture was then homogenized using a rotor stator mixer for 10 minutes at 8000 rpm. The resulting emulsified blend was stirred with low shear rate stirring until cool. Then a preservative, 0.48 grams of Phenonip (available from Clariant International, Ltd.) was stirred into the blend. The blend proved to be stable to separation for more than a month.

The liquid was applied to the knuckles of the left hand of a male volunteer in a thickness that approximated 4 mil wet film thickness. Drying time was about 3 minutes, and resulted in a dry film having excellent flexibility and conformability to the skin. Even on repeated flexing, no cracking or flaking occurred. The film had little noticeable gloss on the skin, and remained on the skin for 2 hours for one application, and overnight on subsequent application. Each film was removed from the skin by gentle rubbing with warm tap water. The underlying skin was smooth and soft to the touch.

### EXAMPLE 29

To a jar was added 92.7 g of the dispersion of Example 2, 4.46 g triethyl citrate, and 7.32 g molten Nutrilayer® phytolipids, available from Eastman Chemical Company, Kingsport, Tenn. The jar contents were stirred at low shear, then heated to 80° C. for about 2 hours, and then subjected to stirring at high shear at 8000 rpm using an Ultraturrax high speed disperser for 5 minutes. The capped jar was then rolled to gently mix the contents until the contents reached room temperature. To 10.5 g of this homogeneous blend was added 0.84 g of 2-ethylhexyl methoxycinnamate UV absorber Uvinul MC-80 from BASF), and the blend was stirred until it was homogeneous. The blend was applied to the skin on the volar forearm whereupon it dried to a film. The film absorbed UV light at both 365 nm and 254 nm, as evidenced by the lack of skin florescence where the film had been applied.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. An aqueous film forming composition comprising:
a) from about 5 to about 40 weight % of a sulfonated or sulfated polymer selected from the group consisting of polyesters, acrylics, hydrid copolymers and mixtures thereof;
b) from about 0.001 to about 40 weight % of an active ingredient or agent;
c) at least one of:
i) up to about 25 weight % of a plasticizer; or
ii) up to about 10 weight % a humectant; and
d) an amount of water so that the sum of the weight percents equals 100, wherein said film forming composition has a viscosity of from about 5 to about 5000 cPs, and wherein said film has a tack-free time of less than about 15 minutes

2. The aqueous composition of claim 1 having from about 5 to about 35 weight % of said polymer.

3. The aqueous composition of claim 1 having from about 10 to about 20 weight % of said polymer.

4. The aqueous composition of claim 1 having from about 0.1 to about 30 weight % of an active ingredient or agent.

5. The aqueous composition of claim 1 having from about 1 to about 15 weight % of the active ingredient.

6. The aqueous composition of claim 1 further comprising up to about 15 weight % of an emulsifier.

7. The aqueous composition of claim 6 having from about 1.5 to about 7 weight % of an emulsifier.

8. The aqueous composition of claim 1 wherein when said polymer has a Tg below about 35° C., and said formulation has from about 0.1 to about 20 weight % of a plasticizer.

9. The aqueous composition of claim 8 wherein the formulation has from about 1 to about 15 weight % of a plasticizer.

10. The aqueous composition of claim 8 wherein the formulation has from about 1 to about 10 weight % of a plasticizer.

11. The aqueous composition of claim 1 wherein the polymer is a sulfopolyester

12. The aqueous composition of claim 1 wherein a film has an elongation of at least 50% when pulled at a rate of 10 inches per minute.

13. The aqueous composition of claim 1 wherein a film has an elongation greater than 200% when pulled at a rate of 10 inches per minute.

14. The aqueous composition of claim 1 wherein the amount of humectant is from about 0.5 to about 10 weight % of the formulation.

15. The aqueous composition of claim 1 wherein the amount of humectant is from about 1 to about 5 weight % of the formulation.

16. The aqueous composition of claim 1 wherein the active ingredient is a pharmaceutically active agent.

17. The aqueous composition of claim 1 wherein the active ingredient is a topical skin agent.

18. The aqueous composition of claim 17 wherein the topical skin agent is selected from the group consisting of aloe, Camellia sinensis (green tea), camomile, ginseng, grape, licorice, cucumber, corn flower, orange peel, dog rose hip, extracts such from seaweed, kelp, and algae, rice bran oil, phytosterols such as dehydro-campesterol, dehydro-sitosterol, B-sitosterol, campesterol, delta-stigmasterol, brassicasterol and stigmasterol, phytosterol esters of two to thirty carbon acids, rice bran phytolipids, palm oil, squalene, coenzyme Q, erucamide, dicaprylyl carbonate, soybean or maleated soybean oil, olive oil, wheat germ oil, caffeine, carnitine, beeswax, paraffin wax, carnauba wax, Shea butter, coco butter, sunflower butter, mango butter, kokaum butter, sal butter, olive butter, vegetable oil butter, glycolic acid, lactic acid, malic acid, and citric acid, salicylic acid, a polymeric hydroxylic acid, β-glucan, corticosteroids, urea, panthenol, an anthocyanidin, a phytic acid, and an amino acid such as glycine, proline, lysine, leucine, alanine, arginine, and serine, avocado oil, nut and berry oils such as almond oil, walnut oil, mineral oil, petrolatum, dimethicone, dimethicone copolyol, natural and synthetic peptides, ubiquinone, hydroxypropyl guar, trimonium chloride, distearyl dimethyl ammonium chloride, sorbitol, glycerin, propylene glycol, ethylene glycol, polyethylene glycol, polypropylene glycol, 1,3-butane diol, hexylene glycol, isoprene glycol, xylitol, fructose or mixtures thereof.

19. The aqueous composition of claim 6 wherein the emulsifier is selected from the group consisting of sodium lauryl sulfate, alcohol ethoxylate sulfate salts, fatty alkyl alcohol, sterol ethoxylates, stearic acid and its salts, glyceryl, diglyceryl, polyglyceryl monoesters of fatty acids, ester ethoxylates, alkyl polyglycosides and saccharide fatty amides, ethoxylate phosphates, fatty alcohol phosphates, non-ionic alcohol ethoxylates or mixtures thereof.

20. The aqueous composition of claim 19 wherein the emulsifier is selected from the group consisting of phosphatidylglycerol, lecithin, stearic acid and its salts, cholesterol ethoxylates, glyceryl stearate, vitamin E ethoxylate, d-alpha-tocopheryl polyethylene glycol-100 succinate, dicetyl phosphate, cetyl phosphate, ceteth-10 or mixtures thereof

21. The aqueous composition of claim 1 wherein the plasticizer is selected from the group consisting of 1,2-propylene glycol, ethylene glycol, 1,3propylene glycol, 2-methyl-1,3-propanediol, butylene glycol, hexanediol, octanediol, glycerin, trihydroxybutane, trihydroxyhexane, alcohols having up to about six hydroxyl groups, diethylene glycol, dipropylene glycol, triethylene glycol, tetraethyene glycol, tripropylene glycol, ethoxylated alcohols, propoxylated alcohols, ethoxylated and propoxylated alcohols, hydroquinone bis(hydroxyethyl ether), cyclohexanol hydroxyethyl ether, sorbitol trihydroxyethyl ether, catechol bis(hydroxyethyl ether), or mixtures thereof.

22. The aqueous composition of claim 1 further comprising less than about 10 weight % of a secondary beneficial ingredient selected from the group consisting of polyvinylpyrrolidinone, silicone oils, ester emollients, cosmetically acceptable hydrocarbons, fatty acids, fatty alcohols, thickeners, preservatives, vitamins, skin lightening agents, desquamation agents, anti-microbial agents, colorants, fragrances, opacifiers, abrasives or scrubbing agents.

23. The aqueous composition of claim 1 wherein said polymer is a hybrid copolymer.

24. The aqueous composition of claim 23 wherein said hybrid copolymer comprises a sulfonated or sulfated polyester and a component comprising at least one acrylic monomer residue.

25. The aqueous composition of claim 24 wherein said sulfonated or sulfated polyester comprises from about 3 to about 95 weight % of said hybrid copolymer.

26. The aqueous composition of claim 24 wherein said sulfonated or sulfated polyester comprises from about 5 to about 80 weight % of said hybrid copolymer.

27. The aqueous composition of claim 24 wherein said sulfonated or sulfated polyester comprises from about 10 to about 60 weight % of said hybrid copolymer.

28. A dermatological acceptable film formed from the composition of claim 1.

29. The film of claim 28 wherein said active agent is fugitive from said film and is selected from the group consisting of aloe, Camellia sinensis (green tea), camomile, ginseng, grape, licorice, cucumber, corn flower, orange peel, dog rose hip, extracts such from seaweed, kelp, and algae, rice bran oil, phytosterols such as dehydro-campesterol, dehydro-sitosterol, B-sitosterol, campesterol, delta-stigmasterol, brassicasterol and stigmasterol, phytosterol esters of two to thirty carbon acids, rice bran phytolipids, palm oil, squalene, coenzyme Q, erucamide, dicaprylyl carbonate, soybean or maleated soybean oil, olive oil, wheat germ oil, caffeine, carnitine, beeswax, paraffin wax, carnauba wax, Shea butter, coco butter, sunflower butter, mango butter, kokaum butter, sal butter, olive butter, vegetable oil butter, glycolic acid, lactic acid, malic acid, and citric acid, salicylic acid, a polymeric hydroxylic acid, β-glucan, corticosteroids, urea, panthenol, an anthocyanidin, a phytic acid, and an amino acid such as glycine, proline, lysine, leucine, alanine, arginine, and serine, avocado oil, nut and berry oils such as almond oil, walnut oil, mineral oil, petrolatum, dimethicone, dimethicone copolyol, natural and synthetic peptides, ubiquinone, hydroxypropyl guar, trimonium chloride, distearyl dimethyl ammonium chloride, sorbitol, glycerin, propylene glycol, ethylene glycol, polyethylene glycol, polypropylene glycol, 1,3-butane diol, hexylene glycol, isoprene glycol, xylitol, fructose or mixtures thereof.

30. The film of claim 28 wherein said active agent is fugitive and from about 1 to about 100% of the fugitive active agent transfers from the film to the epidermis of the user.

31. The film of claim 28 wherein said active agent is fugitive and from about 10 to about 100% of the fugitive active agent transfers from the film to the epidermis of the user.

32. The film of claim 28 wherein said active agent is fugitive and greater than about 80% of the fugitive active agent transfers from the film to the epidermis of the user.

33. The film of claim 28 wherein said active agent is substantially captive in said film and is selected from the group consisting of octyl cinnamate, oxybenzone, 2-ethyl hexyl salicylate, para-amino benzoic acid, menthyl anthranilate, avobenzone, 3-benzylidenebornan-2-one, 5-benzoyl-4hydroxy-4-hydroxy-2-methoxy benzene sulfonic acid, 2-phenyl benzimidazole-5-sulfonic acid, 3,3′-(1,4phenylenedimethylidene)-bis(7,7-dimethyl-2-oxo-bicyclo[2.2.1]heptane-1-methane sulfonic acid) sodium salt or mixtures thereof.

34. The film of claim 33 wherein less than about 25% of the active agent is transferred to the epidermis of the user.

35. The film of claim 34 wherein less than about 15% of the active agent is transferred to the epidermis of the user.

36. The film of claim 34 wherein less than about 5% of the active agent is transferred to the epidermis of the user.

37. The film of claim 28 wherein the amount of humectant is from about 0.5 to about 10 weight % of the formulation.

38. The film of claim 28 further comprising from about 1 to about 10 weight % of an emulsifier.

39. The film of claim 28 having from about 0.1 to about 20 weight % of a plasticizer when said polymer has a Tg above about 35° C.

40. A method for delivering an active agent to the epidermis comprising: providing a film forming composition of claim 1, and applying the formulation to a predetermined area of skin.

41. The method of claim 40 wherein the applying step is selected from brushing, spraying, coating, and spreading.
